(19) United States
(12) Patent Application Publication
(10) Pub. No.: US 2003/0199832 A1
(43) Pub. Date: Oct. 23, 2003

(54) Dosing device with at least two media chambers

(76) Inventors: Juergen Greiner-Perth, Gottmadingen (DE); Peter Stadelhofer, Singen (DE)

Correspondence Address: FLYNN THIEL BOUTELL & TANIS, P.C., 2026 RAMBLING ROAD, KALAMAZOO, MI, 49008-1699, US

(21) Appl. No.: 10/419,469

(22) Filed: Apr. 21, 2003

(30) Foreign Application Priority Data
Apr. 22, 2002 (DE) 102 18 782.7

(51) Int. Cl. A61M 5/00; A61N 1/30; A61B 19/00

(52) U.S. Cl. 604/190; 604/19; 604/181; 604/187; 604/403; 604/257

(57) ABSTRACT

Dosing device with at least two media chambers.

A dosing device with at least two media chambers for storing at least two different media, with a mixing chamber for mixing the media, as well as with pressure generating means for delivering the media mixture from the mixing chamber to at least one outlet is known.

According to the invention, a flow channel (2, 23) connecting at least one media chamber to the mixing chamber is at least zonally constructed both as a supply channel for supplying at least one medium to the mixing chamber and also as a delivery channel for delivering the media mixture to the outlet (17, 27).

The invention relates to a dosing device with at least two media chambers for storing at least two different media, having a mixing chamber for mixing the media and pressure generating means for delivering the media mixture from the mixing chamber to the at least one outlet.

Such a dosing device is known from DE 197 49 514 A1. The dosing device is constructed as a dispenser, which on the one hand has a media chamber for storing a particulate solid material. agent and on the other a further media chamber for storing a carrier liquid with which the particulate solid material agent is mixed prior to discharge. One of the two media chambers additionally serves as a mixing chamber, in that the in each case other medium is supplied to said first media chamber. Subsequently the media mixture is discharged.

The problem of the invention is to provide a dosing device of the aforementioned type, which has a simple construction and variable useability.

This problem is solved in that a flow channel connecting at least one media chamber with the mixing chamber is at least zonally constructed both as a supply channel for supplying at least one medium to the mixing chamber and as a delivery channel for delivering the media mixture in the direction of the outlet. As a result of the solution according to the invention the flow channel takes over a double function, in that it initially ensures the supply of the at least one medium to the mixing chamber. Following the mixing process it additionally serves as a delivery channel for discharging the media mixture. Advantageously it is possible to flow through the flow channel in particular in opposing directions. The solution according to the invention is particularly suitable for the storage and mixing of a particulate, particularly pulverulent or granular and/or lyophilized solid material agent with a corresponding carrier liquid in which the solid material agent either dissolves or is mixed in some other way. The invention is alternatively suitable for the mixing of two liquids, particularly suspensions or solutions, one liquid preferably serving as the active agent carrier. The mixing chamber can either be provided separately with respect to the at least two media chambers, or can be integrated into one of the two media chambers. If more than two media have to be mixed together, a correspondingly larger number of media chambers is available. The solution according to the invention has particular advantages in that from the fluidic standpoint a particularly simple construction can be obtained.

According to a development of the invention pressure means are provided, which in time-displaced manner initiate in the flow channel feed pressures in different directions. Such pressure means have in particular a mechanical construction and are manually operable. It is possible to house at least one medium in a hermetically sealed medium chamber and to pressurize the same, so that at least one media chamber serves as a pressure reservoir.

According to a further development of the invention with the pressure means are associated reversing means for changing the direction of the delivery pressure within the flow channel. For this purpose are in particular provided manually or automatically actuated on/off valves. It is sufficient to position in the dosing device a single, correspondingly adapted on/off valve. As a function of the size and design of the dosing device the reversing means can also be electrically, hydraulically or pneumatically driven.

In a further development of the invention the pressure means are subdivided into first and second pressure generating means acting in directions on the flow channel. It is possible in this development to obviate the need for reversing means, particularly as the pressurizing of the flow channel can take place in different directions in a time-displaced sequence.

According to a further development of the invention at least one media chamber is housed in an ampoule which can be temporarily connected to the flow channel. Thus, said ampoule can be separated from the dosing device and replaced by another ampoule. As a result it is possible to use the dosing device for different pharmaceutical agents. In addition, after use, i.e. particularly after emptying, the ampoule can be removed and disposed of. This more particularly makes it possible to successively connect to the flow channel several media, particularly active agents or carrier liquids, which are in each case housed in their own ampoules, and in particular supply to the mixing chamber. Therefore it is also possible to mix together several active agents and/or carrier liquids or other media, without a correspondingly large number of media chambers having to be made permanently available in the dosing device. It is particularly advantageous if the corresponding ampoules can be applied or used on a corresponding connection or reception area of the dosing device and can be connected to the flow channel. After use the corresponding ampoule can be easily removed again for replacement by a further ampoule. It is obviously also possible to keep the corresponding ampoule fixed to the dosing device following the removal of the medium contained therein in order to keep closed and therefore protected the corresponding connection or reception area.

Further advantages and features of the invention can be gathered from the claims and the following description of preferred embodiments of the invention illustrated by the attached drawings, wherein show:

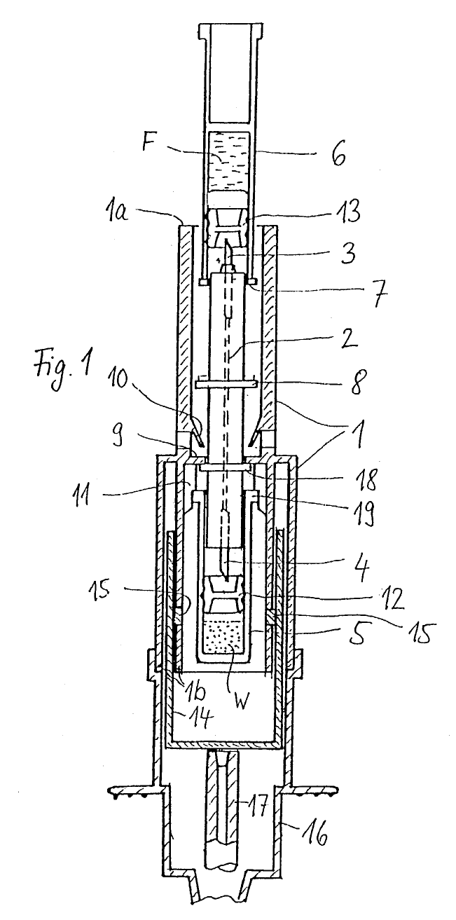

FIG. 1 A sectional view of a first embodiment of a dosing device according to the invention.

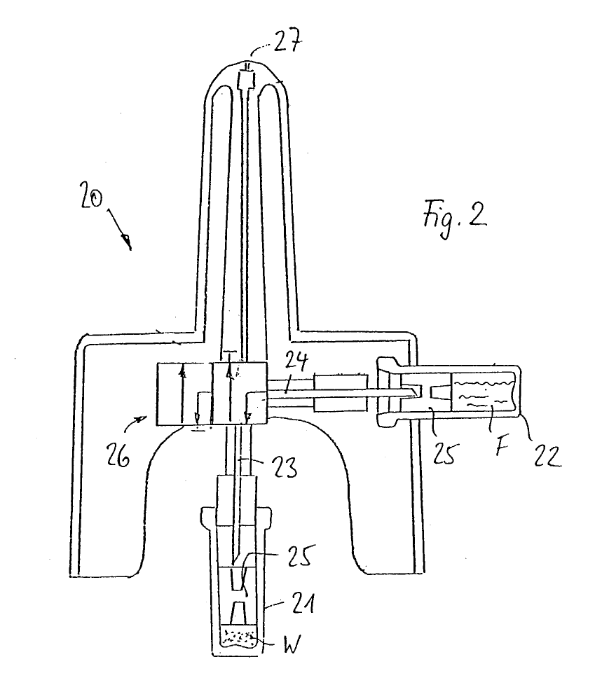

FIG. 2 Diagrammatically in a sectional view a second embodiment of dosing device according to the invention in a first rest position.

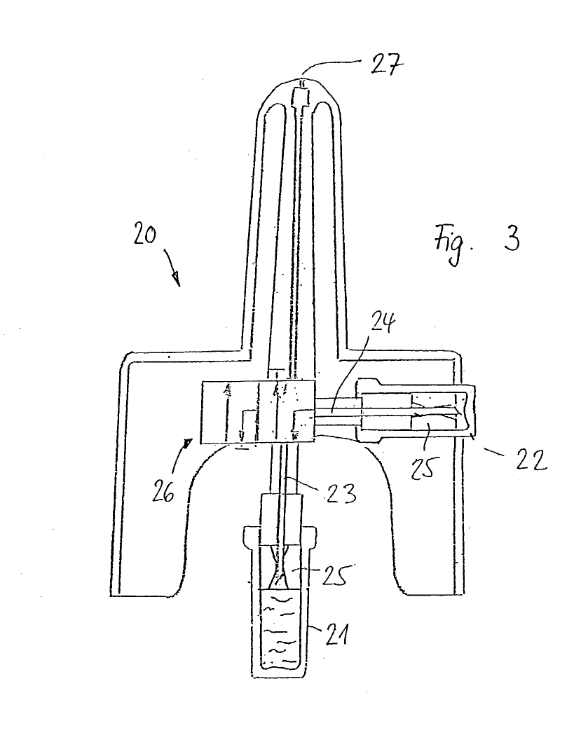

FIG. 3 The dosing device of FIG. 2 in a mixing position.

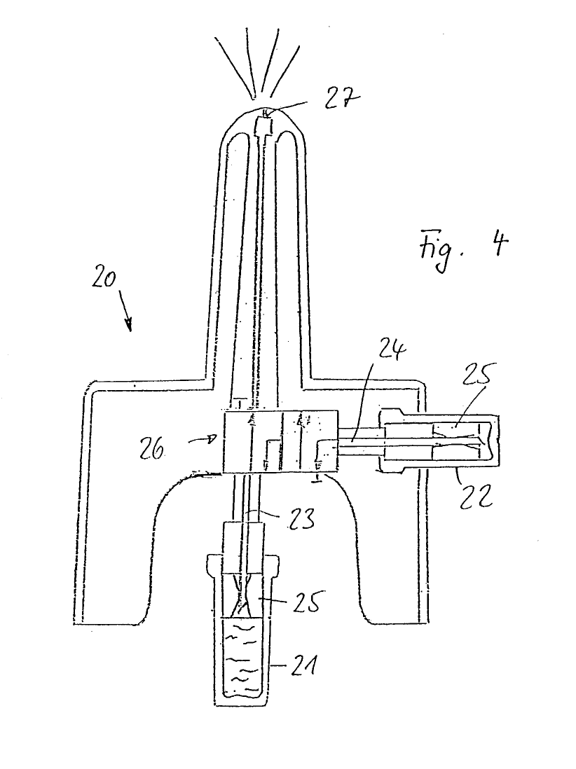

FIG. 4 The dosing device of FIG. 2 in a discharge position.

A dosing device according to FIG. 1 has a casing 1 in which is axially displaceably mounted a cylinder-like filler. The filler is traversed coaxially to its median longitudinal axis by a flow channel 2, which is extended at the opposite front ends of the filler by in each case one cannula 3, 4, which are pressed into the filler. In the orientation of the casing and filler in accordance with FIG. 1, cannula 3 projects upwards and cannula 4 downwards from the filler.

With the filler are associated two ampoules 5, 6 in the vicinity of the opposite front ends and they are in each case mounted on the front end of the filler serving as a connection. The lower ampoule 5 in FIG. 1 serves as a media chamber for a pulverulent, pharmaceutical agent W. The ampoule 5, open to the filler, is sealed by an elastic material stopper 12 immediately following on to the filling volume for the active agent W. The second ampoule 6, which forms a media chamber for a carrier liquid F, is mounted on the upper front end of the filler and projects upwards over an open frontal area 1a of casing 1. The media chamber of ampoule 6 is also sealed by an elastic material stopper 13.

A lower area 1b of the casing 1 has a outer, cylindrical wall and coaxially thereto and inwardly displaced and spaced therefrom an inner, cylindrical wall. On the inner, cylindrical wall is located a cylindrical actuator 14, which is fixed with the aid of tear-off webs 15 to the inner, cylindrical wall of the casing 1.

A nose spray attachment 16 is detachably mounted on the outer frontal edge of the area 1b of casing 1 and in said attachment is integrated a discharge channel 17 terminating in a not shown discharge opening.

In the position shown in FIG. 1 the nose spray attachment 16 forms an actuation protection for the actuator 14 and this more particularly represents the delivery state of the dosing device. In the delivery state the ampoule 6 provided with the carrier liquid F is separately delivered. For mixing the carrier liquid F with the active agent W the ampoule 6 is mounted in and on the filler in the upper frontal area 1a of the casing 1. The ampoule 6 engages on a tear-off ring 7. The filler is displaced axially downwards on axially pressing in the ampoule 6. The ampoule 5 with the active agent W has on its upper edge a collar 19, which is held positively or non-positively up to a given axial loading between the ribs 11 of casing 1. As a result the needle-like cannula 4 perforates the stopper 12 until a lower frontal edge of the filler engages on the stopper 12. Additionally the stopper 12 is slightly downwardly displaced, preferably by approximately 0.5 mm. This makes it possible to release a sticking of the stopper due to long-term storage. In this position a collar 18 of the filler engages on the upper frontal edge of the ampoule 5. On further downwardly pressing the filler, said collar 18 presses the ampoule 5 out of the ribs 11. The ampoule 5 cannot drop downwards, because it is held on the filler by the frictional connection of the cannula 4 inserted in the stopper 12. The downward axial displacement of the filler is continued until a further support 8 of the filler engages on a casing-side annular shoulder 9. With the annular shoulder 9 are associated clip-like pull-back stops 10, which axially fix the collar 8 on the annular shoulder 9.

In the case of a constant, further compressive loading of the upper ampoule 6 in the downwards direction, the tear-off ring 7 tears off and the ampoule 6 is engaged axially on the filler. The upper, needle-like cannula 3 perforates the stopper 13 of ampoule 6 and penetrates the media chamber filled with the carrier liquid F. As a result of the constant, further downward pressing of the ampoule 6, the carrier liquid F is forced through the flow channel 2 downwards into the media chamber filled with the pulverulent agent W. As a result of the volume increase in the media chamber of ampoule 5 due to the pressing in of the carrier liquid F, ampoule 5 is moved axially downwards until it engages on the bottom of the actuator 14. Thus, the media chamber of ampoule 5 serves as a mixing chamber for mixing the carrier liquid F with the pulverulent active agent W. This terminates the mixing process.

The emptied ampoule 6 is now extracted from the casing 1 and removed. The nose spray attachment 16 is drawn off in the vicinity of the actuator 14 and engaged on the upper frontal area 1a of the casing 1. Necessarily the discharge channel 17 is mounted on the filler cannula 3, so that the discharge channel 17 is connected to the flow channel 2. This gives the functional position of the dosing device in which dosing of the media mixture can take place by means of the actuator 14. The actuation principle of the actuator 14 is not illustrated in detail and will not be described here, because the prior art adequately discloses various actuating principles for single or multiple dosing with specified dosing volumes. As a function of requirements the expert will choose the most suitable actuating mechanism and use it in the present dosing device.

The dosing device 20 according to FIGS. 2 to 4 has a not further designated casing, which is provided with two connections for the engagement of in each case one ampoule 21, 22. Within each connected is in each case integrated a channel 23, 24, channel 23 forming a flow channel in the sense of the invention and channel 24 a supply channel. In accordance with FIGS. 2 to 4, an ampoule 22 provided with a pharmaceutical agent W and which is sealed by a stopper 25, is engaged from below on the correspondingly downwardly projecting connection, whereas the other ampoule 21 provided with a carrier liquid F is mounted from the side on the laterally projecting connection. Ampoule 21 is also sealed with a stopper 25.

In a first step both ampoules 21, 22 are mounted on the connections to such an extent that needle-like cannulas of channels 23, 24 of the connections perforate the stopper 25. The two channels 23, 24 are interconnected by means of a reversing valve 26, constructed here as a 3/2-way valve. In the position of the reversing valve 26 shown in FIG. 2 the two channels 23, 24 are interconnected. By pressing in the ampoule 22, the carrier liquid contained therein is pumped into the lower ampoule 21 provided with the pharmaceutical agent. On pressing the carrier liquid F into ampoule 21 the latter is moved downwards. It is alternatively possible to move the stopper 25 upwards. In both cases the necessary volume increase within the ampoule 21 is obtained.

The corresponding media mixture, which can be in the form of a solution or dispersion, is shown in FIG. 3. In order now to discharge outwards through outlet 27 the media mixture from ampoule 21, the reversing valve 26 is reversed in such a way that the flow channel 23 is connected to an outlet channel issuing into the outlet 21. Through upward pressure on the ampoule 21 the corresponding pumping movement is applied in order to discharge the media mixture through the flow channel 23 and the outlet channel. Thus, the flow channel 23 is supplied with a flow in the opposite direction (FIG. 4).

According to a not shown embodiment of the invention it is possible to provide the dosing device 20 with an inspection window in the vicinity of the ampoule 21 in order to be able to monitor the result of the thorough mixing of both media.

Apart from the described manual switching of the reversing valve 26, it is also possible to bring about a forced, pressure-dependent switching or indexing, so that there is a self-indexing of the reversing valve 26. The self-indexing can also take place through other forms of control elements, which are in particular coupled in movement-dependent manner to the volume change within the ampoule 21. There can also be a time-dependent self-indexing.

Besides the described, simple pump actuation, the media mixture can also be discharged through other, fundamentally known types of actuating mechanisms in single or multiple stroke form. After use, both ampoules 21 and 22 can be removed again from the corresponding connections and replaced by new ampoules. After pumping the carrier liquid F from the ampoule 22, it is also possible to remove the ampoule 22 from the connection and to engage a further ampoule with an additional active liquid agent or another carrier liquid, so as to also pump round the latter in the media chamber of the lower ampoule 21 serving as a mixing chamber. In this case the lower ampoule 21 must be sufficiently large to permit it to receive said liquid volume.

1. Dosing device with at least two media chambers for storing at least two different media, with a mixing chamber for mixing the media, as well as pressure generating means for delivering the media mixture from the mixing chamber to the at least one outlet, wherein a flow channel (2, 23) connecting at least one media chamber to the mixing chamber is at least zonally constructed both as a supply channel for supplying at least one medium to the mixing chamber and as a delivery channel for delivering the media mixture in the direction of the outlet (17, 27).

2. Dosing device according to claim 1, wherein pressure means are provided, which in time-displaced manner initiate delivery pressures in different directions in the flow channel (2, 23).

3. Dosing device according to claim 2, wherein the pressure means are subdivided into first and second pressure generating means acting in different directions on the flow channel (2, 23).

4. Dosing device according to claim 2, wherein with the pressure means are associated reversing means (26) for changing the direction of the delivery pressure within the flow channel (23).

5. Dosing device according to claim 4, wherein a 3/2-way valve (26) is associated as the reversing means with the flow channel (23).

6. Dosing device according to claim 1, wherein one medium chamber for storing a medium is constructed as a mixing chamber.

7. Dosing device according to claim 1, wherein one medium chamber is designed for storing a particulate solid medium (W), particularly a pharmaceutical agent.

8. Dosing device according to claim 1, wherein the other medium chamber is provided for storing a carrier liquid (F) for the solid medium (W).

9. Dosing device according to claim 1, wherein at least one medium chamber is housed in an ampoule which can be temporarily linked with the flow channel (2, 23).
